(19) United States
(12) Patent Application Publication
(10) Pub. No.: US 2024/0047010 A1
(43) Pub. Date: Feb. 8, 2024

(54) STRUCTURAL VARIANT EVALUATION THROUGH ITERATIVE GENOME CONSTRUCTION

(71) Applicant: GOOGLE LLC, Mountain View, CA (US)

(72) Inventors: Ali BASHIR, San Mateo, CA (US); Zahra SHAMSI, Mountain View, CA (US); Wesley Wei QIAN, Austin, TX (US); Tze Way Eugene IE, Los Altos, CA (US); Jeffrey CHAN, New York, NY (US); Marc BERNDL, Mountain View, CA (US); Martin MLADENOV, Carapicuiba, Sao Paulo (BR); Lawrence Stephen LANSING, Los Gatos, CA (US)

(21) Appl. No.: 18/265,795

(22) PCT Filed: Feb. 1, 2022
(86) PCT No.: PCT/US2022/014764

§ 371 Date: Jun. 7, 2023

Related U.S. Application Data
(60) Provisional application No. 63/144,235, filed on Feb. 1, 2021.

(51) Int. Cl. G16B 30/10

(52) U.S. Cl. CPC G16B 30/10

(57) ABSTRACT

A method is provided for determining a sample genome from a plurality of read fragments and a reference genome. The method includes: (i) applying a first putative variant event, selected from a set of candidate variant events, to the sample genome to update the sample genome; (ii) mapping the plurality of read fragments to the updated sample genome; (hi) based on the mapping of the plurality of read fragments to the updated sample genome, determining a first read mapping cost function; and (iv) based on the first read mapping cost function, retaining the updated sample genome and removing the first putative variant event from the set of candidate variant events.

### CROSS-REFERENCE TO RELATED APPLICATION

The present application claims priority to U.S. Provisional Patent Application No. 63/144,235, filed on Feb. 1, 2021, the contents of which are hereby incorporated by reference.

### BACKGROUND

Early DNA sequencing techniques, such as chain-termination methods, provided reliable solutions for reading individual DNA fragments See Sanger, F. et. al. (1977) DNA sequencing with chain-terminating inhibitors. Proc. Natl. Acad. Sci. U.S.A. 74, 5463-5467. While these first-generation technologies are effective for sequencing target genes, applying them to sequencing entire chromosomes or genomes is costly and expensive. For example, the first sequencing of a human genome—which was accomplished using the Sanger method—cost hundreds of millions of dollars and took over a decade to complete. This high cost was largely due to the sequential nature of first-generation sequencing methods; each fragment had to be individually read and manually assembled to construct a full genome.

Next generation sequencing (NGS) technologies have significantly reduced the cost of DNA sequencing by parallelizing DNA fragment reading. Some NGS methods are capable of performing millions of sequence reads concurrently, generating data for millions of base pairs in a matter of hours. See Hall, N. (2007) Advanced sequencing technologies and their wider impact in microbiology. The Journal of Experimental Biology, 209, 1518-1525. Many NGS technologies have been proposed, and employ various chemical processes, use varying read lengths, and have demonstrated various ranges of accuracy. See Metzker, M. (2010) Sequencing technologies—the next generation. Nature Reviews, Genetics, Volume 11, 31-46; see also Shendure, J. et. al. (2008) Next-generation DNA sequencing. Nature Reviews, Biotechnology, Volume 26, Number 10, 1135-1145.

NGS methods generally involve separating a DNA sample into fragments and reading the nucleotide sequence of those fragments in parallel. The resulting data generated from this process includes read data for each of those fragments, which contains a continuous sequence of nucleotide base pairs (G, A, T, C). However, while the arrangement of base pairs within a given fragment read is known, the arrangement of the fragment reads with respect to each other is not. Thus, to determine the sequence of a larger DNA strand (such as a gene or chromosome), read data from multiple fragments must be aligned. This alignment is relative to other read fragments, and may include overlapping fragments, depending upon the particular NGS method used. Some NGS methods use computational techniques and software tools to carry out read data alignment.

Accurate sequence read alignment is the first step in identifying genetic variations in a sample genome. The diverse nature of genetic variation can cause alignment algorithms and techniques to align sequence reads to incorrect locations within the genome. Furthermore, the read process used to generate sequence reads may be complex and susceptible to errors. Thus, many sequence read alignment techniques can misalign a sequence read within a genome, which can lead to incorrect detection of variants in subsequent analyses.

Once the read data has been aligned, that aligned data may be analyzed to determine the nucleotide sequence for a gene locus, gene, or an entire chromosome. However, differences in nucleotide values among overlapping read fragments may be indicative of a variant, such as a single-nucleotide polymorphism (SNP) or an insertion or deletion (INDELs), among other possible variants. For example, if read fragments that overlap at a particular locus differ, those differences might be indicative of a heterozygous SNP. As another example, if overlapping read fragments are the same at a single nucleotide, but differ from a reference genome, that gene locus or gene may be a homozygous SNP with respect to that reference genome. Accurate determination of such variants is an important aspect of genome sequencing, since those variants could represent mutations, genes that cause particular diseases, and/or otherwise serve to genotype a particular DNA sample.

The demand for high efficiency and low-cost DNA sequencing has increased in recent years. Although NGS technologies have dramatically improved upon first-generation technologies, the highly-parallelized nature of NGS techniques has presented challenges not encountered in earlier sequencing technologies. Errors in the read process can adversely impact the alignment of the resulting read data, and can subsequently lead to inaccurate sequence determinations. Furthermore, read errors can lead to erroneous detection of variants.

A more comprehensive and accurate understanding of both the human genome as a whole and the genomes of individuals can improve medical diagnoses and treatment. NGS technologies have reduced the time and cost of sequencing an individual's genome, which provides the potential for significant improvements to medicine and genetics in ways that were previously not feasible. Understanding genetic variation among humans provides a framework for understanding genetic disorders and Mendelian diseases. However, discovering these genetic variations depends upon reliable read data and accurate read sequence alignment. In particular NGS technologies, which are based on short reads, struggle to detect so-called structural variants, which can affect an extended genome region e.g. 100s of bases or more.

### SUMMARY

The process of determining a genome or other sequence data from a sample (e.g., from a set of read fragments determined from the sample) is complex and may include a variety of processes. Such sequence data is referred to herein as a “sample genome,” which as used herein can include sequence information for an individual organism's entire genome, for a single chromosome or set of chromosomes, for a region of a chromosome (e.g., a gene locus), or sequence information for some other subset of the total genomic sequence data for the organism.

Determination of such a sample genome can provide a variety of benefits. Determining a sample genome for an individual (e.g., from cells explanted from a tumor or other specific tissue of the individual) can be useful to identify predisposition toward disease(s), the existence or progression of a disease, to determine a course of treatment (e.g., to identify the most efficacious and/or least harmful drugs or other treatments), or to provide a number of other preventive and/or curative benefits to the individual. Determining sample genomes for multiple individual organisms can also facilitate determining the genetic makeup of one or more populations of organisms, allowing for the determination of historical development of the population(s) or other information about the current or historical genetics of the population(s).

Sequencing of genetic material in a sample often results in a plurality of short (e.g., dozens to thousands of base-pairs long) read fragments that reflect respective portions of the sequence of the genetic material. The relative locations and/or degrees of overlap of the read fragments may be totally unknown, or some amount of distal information may be available. For example, information about the relative location or read direction of pairs or other sets of the read fragments could be available, e.g., as a result of a paired-end sequencing technique. Thus, a variety of computational techniques may be applied (e.g., alignment of fragments, statistical inference) to determine the underlying sequence that resulted in the observed set of read fragments.

Embodiments described herein provide improved sample genome determination by leveraging known information about the baseline genome and known genetic variants thereof for the species of a target organism. These embodiments include iteratively applying known or inferred structural variants (e.g., indels, tandem repeats, transpositions, inversions, copy-number variants, etc.) to a reference genome to iteratively generate the sample genome for an individual. Each time one or more structural variants is applied to the putative sample genome, the observed read fragments from the individual are re-mapped and the quality of the mapping (e.g., the likelihood of the putative sample genome, given the observed set of read fragments) assessed in order to determine whether to retain the added one or more structural variants (and, potentially, to continue the iterative process by adding additional structural variant(s)) or to discard it/them in favor of alternative structural variants. More particularly, embodiments of the techniques described herein leverage information from longer sequences, which are difficult and costly to obtain, to enable NGS-based technologies to be used to accurately detect variants, in particular structural variants.

Such processes as are described herein lead to improved sample genomes and reduced computational costs. This can include reducing the amount of time and/or memory required to generate an estimated genome. Such a reduction in memory and/or time required can be related to the described genome reconstruction methods being iterative, with individual putative events being selected accordingly a statistically-driven process and applied to the iteratively-generated sample genome, and evaluated, individually and in order. Thus, each putative event insertion and evaluation only requires assessing, and potentially re-positioning, a limited number of the available sequence reads that correspond to the location of the putative event and/or to the sequence of a DNA segment inserted/removed/translocated as part of the putative event. If the putative event is discarded, the computational cost of removing the putative event from the genome being reconstructed may be similarly computationally inexpensive, and may be effected relatively cheaply without storing, and reverting to, a full record of the genome prior to insertion of the putative event. Indeed, the local nature of the insertion of most putative events could allow the methods described herein to be performed with multiple putative events inserted and evaluated by respective computational instances (e.g., virtual machines, servers, blades of a server) functionally in parallel by segregating portions of the genome being reconstructed across multiple different computational instances (e.g., with respective regional sub-sections of the genome and the available sequence reads resident in memory only for one or a few instances that are relevant to the regional sub-sections).

Accordingly, in a first aspect a method for determining a sample genome from a plurality of read fragments and a reference genome is provided, the method including: (i) applying a first putative variant event, selected from a set of candidate variant events, to a sample genome based on the reference genome to update the sample genome; (ii) mapping the plurality of read fragments to the updated sample genome; (iii) based on the mapping of the plurality of read fragments to the updated sample genome, determining a first read mapping cost function; and (iv) based on the first read mapping cost function, retaining the updated sample genome and removing the first putative variant event from the set of candidate variant events.

The read fragments are obtained by sequencing genetic material. To arrive at a sample genome that accurately reflects the genome of a sample from which the read fragments were obtained, the steps of the method can be performed repeatedly, beginning with an initial version of the sample genome, e.g. beginning with the reference genome. The method may continue by progressively applying putative variant events, from the set of candidate variant events, to the sample genome. In some repetitions of the steps of the method, an applied putative variant event may be rejected based on a read mapping cost function, in which case the applied putative variant event may be removed from the updated sample genome and/or the updated sample genome may be discarded in favor of a version of the sample genome prior to application of the applied putative variant event. Rejecting (or retaining) an updated sample genome based on a read mapping cost function can include comparing a value of the read mapping cost function to a threshold value (e.g., determining that the value of the read mapping cost function is greater than, or less than, the threshold value).

A “variant event” may include any of a variety of different variations that may be present in a sample genome. Such a variety event may include single or multiple base insertions/deletions (e.g., novel sequence insertions), transpositions, inversions, copy number variations, tandem duplications/repeats, non-tandem or dispersed duplications/repeats, indels, inversions, or translocations.

The set of candidate variant events can include events from a database of experimentally observed structural variations, e.g., the databases created by the Genome in a Box (GIAB) and/or Human Genome Structural Variation (HGSV) consortiums. Additionally or alternatively, the set of candidate variant events can include at least one variant event determined from the plurality of read fragments, e.g., using a variant calling program.

The method can additionally include, subsequent to retaining the updated sample genome and removing the first putative variant event from the set of candidate variant events: (i) applying a second putative variant event, selected from the set of candidate variant events, to the updated sample genome to further update the sample genome; (ii) mapping the plurality of read fragments to the further updated sample genome; (iii) based on the mapping of the plurality of read fragments to the further updated sample genome, determining a second read mapping cost function; and (iv) based on the second read mapping cost function, rejecting the further updated sample genome. Rejecting the further updated sample genome based on the second read mapping cost function can include comparing a value of the second read mapping cost function to a threshold value (e.g., determining that the value of the second read mapping cost function is greater than, or less than, the threshold value).

Determining the second read mapping cost function can include evaluating a cost function that includes a term that is related to (e.g., dependent on) at least one of: (i) a likelihood of the second putative variant event appearing in a demographic group that includes a subject from which the read fragments were obtained, or (ii) a likelihood of the likelihood of the second putative variant event appearing in a genome given that the first putative variant event appears in the genome.

The method can additionally include, subsequent to retaining the updated sample genome and removing the first putative variant event from the set of candidate variant events: (i) updating at least one of an alignment or a size of the first putative variant event within the updated sample genome to further update the sample genome; (ii) mapping the plurality of read fragments to the further updated sample genome; (iii) based on the mapping of the plurality of read fragments to the further updated sample genome, determining a third read mapping cost function; and (iv) based on the third read mapping cost function, retaining the further updated sample genome. Alternatively, the further updated sample genome may be rejected based on the third read mapping cost function, in which case the applied update to the size and/or alignment of the first putative variant event may be removed from the further updated sample genome and/or the further updated sample genome may be discarded in favor of a version of the updated sample genome prior to updating the size/location of the first putative variant event. Rejecting (or retaining) the further updated sample genome based on the third read mapping cost function can include comparing a value of the third read mapping cost function to a threshold value (e.g., determining that the value of the third read mapping cost function is greater than, or less than, the threshold value).

Updating an alignment of the first putative variant event within the updated sample genome can include shifting the location of the first putative variant event within the updated sample genome and/or shifting a location, within the non-updated sample genome, at which a portion of the non-updated sample genome was copied or removed in order to provide a sequence of the first putative variant event. Updating a size of the first putative variant event within the updated sample genome can include altering a number of repeats of a motif within the first putative variant (e.g., a number of trinucleotide repeats in the first putative variant event) or altering a number of bases copied and/or removed from the non-updated sample genome in order to provide a sequence of the first putative variant event.

Determining the first read mapping cost function can include determining, for at least one region of the updated sample genome, a degree of correspondence between a specified reference distribution (e.g., Poisson, Gaussian, or some other distribution that can correspond to the expected distribution of the coverage depending on the fragmentation, priming, amplification, sequencing, and/or other processes used to generate the read fragments from a sample) and a distribution of coverage of the updated sample genome by the plurality of read fragments after mapping the plurality of read fragments to the updated sample genome. “Coverage” of a particular nucleotide location within the updated sample genome refers to the number of the read fragments that include the particular nucleotide location after the plurality of read fragments have been mapped to the updated sample genome. The “distribution of coverage” of the updated sample genome is the distribution of the coverage values for all, or some sample of, the nucleotide locations within the updated sample genome.

Determining the first read mapping cost function can include determining a number of read fragments of the plurality of read fragments that are not mapped to the updated sample genome after mapping the plurality of read fragments to the updated sample genome.

Determining the first read mapping cost function can include determining a number of read fragments of the plurality of read fragments that are partially mapped to at least one location within the updated sample genome after mapping the plurality of read fragments to the updated sample genome.

Determining the first read mapping cost function can include evaluating a cost function that includes at least one of: (i) a term that is related to (e.g., dependent on) the number of variant events applied to the reference genome to generate the updated sample genome or (ii) a term that is related to (e.g., dependent on) a number of read fragments that are mapped to an event from the set of candidate variant events that is not represented in the sample genome.

Determining the first read mapping cost function can include determining, based on a region of the updated sample genome that is affected by application of the first variant event, a local effect on a global read mapping cost function. Such a local effect could include an effect on the number of read fragments that are now mapped to the affected region that were not previously mapped, or that were mapped to other regions of the sample genome. Such a local effect could include an effect on the number of read fragments that are no longer mapped to the affected region that were previously mapped. Such a local effect could include an effect on an overall coverage distribution of the sample genome due to changes in the coverage of the affected region.

The method could additionally include, prior to applying the first putative variant event to the sample genome to update the sample genome, determining a set of pre-alignments for the plurality of read fragments by determining one or more possible alignments for each read fragment to one or more locations on the reference genome or to one or more locations on one or more of the variant events in the set of candidate variant events. A possible alignment for a read fragment is an alignment of all or a portion of the read fragment to a particular location within the reference genome or a particular location within one of the variant events in the set of candidate variant events. Mapping the plurality of read fragments to the updated sample genome can include selecting, for a particular read fragment in the plurality of read fragments, one of the possible alignments for the particular read fragment. The method could yet further include determining, based on the set of pre-alignments, that a first set of read fragments within the plurality of read fragments and a first set of variant events within the set of candidate variant events are connected via possible alignments to each other but not to other read fragments within the plurality of read fragments or to other variant events within the set of candidate variant events, wherein the first putative variant event is selected from first set of variant events, and wherein mapping the plurality of read fragments to the updated sample genome comprises mapping the first set of read fragments to the updated sample genome. The method could additionally or alternatively yet further include, prior to applying the first putative variant event to the sample genome to update the sample genome, removing from the set of candidate variant events any variant events for which the set of pre-alignments includes no possible alignments with any read fragments. Optionally the pre-alignments may be used in a parallelized implementation of the method, to distribute tasks across computer hardware to be executed in parallel, e.g. for parallel computation of one or more of the cost functions by separating computation of a cost function into sub-computations based on the pre-alignments.

The method could additionally include: (i) applying a filter to determine a second set of variant events that are highly likely to be represented in the sample genome and a third set of variant events that are highly unlikely to be represented in the sample genome; (ii) prior to applying the first putative variant event to the sample genome to update the sample genome, applying the variant events of the second set of variant events to the reference genome to generate the sample genome; and (iii) prior to applying the first putative variant event to the sample genome to update the sample genome, removing the third set of variant events from the set of candidate variant events. The filter can be or include a classifier, e.g. a classifier trained using machine learning. Determining the second set of variant events that are highly likely to be represented in the sample genome can include determining variant events that are expected, with greater than a first threshold confidence level (e.g., 80%, 90%), to be represented in the sample genome. Determining the third set of variant events that are highly unlikely to be represented in the sample genome can include determining variant events that are expected, with greater than a second threshold confidence level (e.g., 80%, 90%, 95%, 99%), not to be represented in the sample genome.

Applying the filter to determine the second and third sets of variant events can include determining, for each of the variant events in the set of candidate variant events, at least one of: (i) the existence of a positive signature read within the plurality of signature reads, or (ii) the existence of a negative signature read within the plurality of signature reads. Determining the existence of a positive signature read within the plurality of signature reads for a particular variant event comprises determining that there exists, within the plurality of read fragments, a read fragment whose existence makes it highly likely that the particular variant event is represented in the sample genome. Determining that there exists, within the plurality of read fragments, a read fragment whose existence makes it highly likely that the particular variant event is represented in the sample genome can include determining that the particular variant event is expected, with greater than a threshold confidence level (e.g., 80%, 90%), to be represented in the sample genome. Determining the existence of a negative signature read within the plurality of signature reads for a particular variant event can include determining that there exists, within the plurality of read fragments, a read fragment whose existence makes it highly likely that the particular variant event is not represented in the sample genome. Determining that there exists, within the plurality of read fragments, a read fragment whose existence makes it highly likely that the particular variant event is not represented in the sample genome can include determining that the particular variant event is expected, with greater than a threshold confidence level (e.g., 80%, 90%), not to be represented in the sample genome.

The method could additionally include generating the plurality of read fragments from a biological sample. This can include applying automated laboratory systems and methods (e.g., next-generation sequencing systems/methods) or some other sequencing experimental procedure to obtain a plurality of DNA (or RNA or other polynucleotide or polypeptide of interest) sequencing read fragments.

The method could be performed as part of an industrial sequence pipeline. For example, the plurality of read fragments could be generated by a first aspect of the industrial sequence pipeline, alignment (and re-alignment) of the read fragments with a putative sample genome before and/or after insertion of a candidate event therein could be performed by a second aspect of the industrial sequence pipeline, etc.

The method could be performed as part of an industrial quality control process for sequencing. For example, the method could be used to generate a sample genome for an input sample based on a plurality of read fragments generated from the input sample. The generated sample genome could then be used to determine whether the input sample was acceptable in some way (e.g., an overall likelihood of the sample genome and/or a minimum likelihood of one or more candidate events inserted therein could exceed a specified threshold). This could include (i) performing the method, (ii) based on the updated sample genome, determining that an input sample from which the plurality of read fragments were generated is suspect, and (iii) responsive to determining the input sample is suspect, generating an additional plurality of read fragments for an additional sample and performing the method based on the additional plurality of read fragments. Determining that an input sample from which the plurality of read fragments were generated is suspect could include determining that an overall likelihood of the sample genome (determined, e.g., from a product of individual likelihoods of each candidate event inserted to generate the sample genome) is less than a threshold value, determining that a minimum likelihood of one or more candidate events inserted to generate the sample genome is less than a specified value, determining that more than a threshold number of the inserted candidate events have likelihoods less than a threshold value, and/or performing some other determination based at least on a sample genome generated for the input sample using the method.

Input samples that are deemed unacceptable, based sample genomes determined therefor using the method, could be re-run. This could include by re-generating the sample via a source chemical or biological process, re-accessing the sample from a human or animal body or other source of biological samples, and/or re-performing one or more processes used to generate, from an input sample, a plurality of read fragments (e.g., cell lysis, DNA amplification, DNA fragmentation, DNA fixation on solid supports, DNA sequence-by-synthesis).

In another aspect, non-transitory computer readable medium is provided, having stored therein instructions executable by a computing device to cause the computing device to determine a sample genome according to the method of the first aspect.

In another aspect, a system is provided that includes one or more processors and a computer-readable medium (e.g., a non-transitory computer readable medium) having stored therein instructions executable by the system to cause the one or more processors to determine a sample genome according to the method of the first aspect.

The system could be, or could form a part of, a sequencing system configured to, among other things, generate a plurality of read fragments from an input sample (e.g., by performing PCR, cell lysis, iterative sequencing-by-synthesis or other fragment sequencing methods, filtering to reject erroneous read fragments or otherwise improve a set of read fragments) and to apply the methods of the first aspect to generate, from the read fragments, a sample genome for the input sample. For example, a sequencer is provided that includes (i) one or more processors, (ii) sequencing means configured to receive an input sample, and (iii) a computer-readable medium having stored therein instructions executable by the system to cause the one or more processors to: (a) operate the sequencing means to generate a plurality of read fragments from the input sample, and (b) based on the generated plurality of read fragments, determine a sample genome for the input sample according to the method of the first aspect.

The foregoing summary is illustrative only and is not intended to be in any way limiting. In addition to the illustrative aspects, embodiments, and features described above, further aspects, embodiments, and features will become apparent by reference to the figures and the following detailed description and the accompanying drawings.

### BRIEF DESCRIPTION OF THE FIGURES

FIG. 1A illustrates an example reference genome and a plurality of read fragments aligned thereto.

FIG. 1B illustrates an example sample genome and a plurality of read fragments aligned thereto.

FIG. 2 illustrates aspects of an example method for determining a sample genome.

FIG. 3A illustrates aspects of an example method for determining a sample genome.

FIG. 3B illustrates aspects of an example method for determining a sample genome.

FIG. 3C illustrates aspects of an example method for determining a sample genome.

FIG. 4 illustrates a flowchart of an example method.

FIG. 5 illustrates an example event decomposition graph.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

### I. INTRODUCTION

Next generation sequencing (NGS) has dramatically reduced the time and cost required to sequence an entire genome. Previous techniques involved sequentially reading out DNA fragments and having trained biochemists arrange the read data to determine the sequence of entire chromosomes. NGS technologies parallelize the sequencing process, allowing millions of DNA fragments to be read simultaneously. Automated computational analyses then attempt to align the read data to determine the nucleotide sequence of a gene locus, gene, chromosome, or entire genome.

The increasing prevalence of NGS technologies has generated a substantial amount of genome data. Analysis of this genome data-both for an individual sample and for multiple samples—can provide meaningful insights about the genetics of a sample (e.g., an individual human patient) or species. Variations between genomes may correspond to different traits or diseases within a species. Variations may take the form of single nucleotide polymorphisms (SNPs), insertions and deletions (INDELs), and structural differences in the DNA itself such as copy number variants (CNVs) and chromosomal rearrangements. By studying these variations, scientists and researchers can better understand differences within a species, the causes of certain diseases, and can provide better clinical diagnoses and personalized medicine for patients.

Many different read processes may be used to generate DNA fragment read data of a sample. Each read process may vary by read length, amplification method, materials used, and the technique used (e.g., chain termination, ligation, etc.). The nature and source of the errors of each read process may vary. Thus, the features that distinguish incorrect alignments, invalid variant calls, or false positive variant detections may differ among read processes.

Note that a “sample” may be a sample from a biological organism (e.g., a human, an animal, a plant, etc.) and/or may be a sample containing synthetic contents. For example, the sample could contain synthetic DNA (or RNA, or some other synthetic polynucleotide) created, e.g., to store information in the sequence or other characteristics of the synthetic DNA. Accordingly, the methods described herein could be applied to extract the information stored in such a sample.

### II. TERMINOLOGY

A. Next Generation Sequencing (NGS)

NGS generally refers to DNA sequencing techniques that involve sequencing multiple DNA fragments of a sample in parallel. The output data may contain nucleotide sequences for each read, which may then be assembled to form longer sequences within a gene, an entire gene, a chromosome, or a whole genome. The specific aspects of a particular NGS technique may vary depending on the sequencing instrument, vendor, and a variety of other factors. Secondary analyses may then involve aligning/assembling the reads to generate a predicted target sequence, detecting variants within the sample, etc.

An example polynucleotide (e.g., DNA) sequencing pipeline may include polynucleotide sequencing (e.g., using one or more next-generation DNA sequencers), read data alignment, variant calling, and/or other methods (e.g., the sample genome determination by iterative event insertion methods described herein). As described herein, a “pipeline” may refer to a combination of hardware and/or software that receives an input material or data and generates a model or output data. The example pipeline receives a polynucleotide-containing sample as input, which is sequenced by polynucleotide sequencer(s) to output read data. Read data alignment occurs by receiving the raw input read data and generating aligned read data. Variant calling can then proceed by analyzing the aligned read data and outputting potential variants. Determination of a sample genome or of some other sequence within the sample, using the methods described herein or other methods, can also then proceed with or without variant calling.

The input sample may be a biological sample (e.g., biopsy material) taken from a particular organism (e.g., a human). The sample may be isolated DNA, RNA, or some other polynucleotide and may contain individual genes, gene clusters, full chromosomes, or entire genomes. Polynucleotides of interest in a sample can include natural or artificial DNA, RNA, or other polynucleotide formed of some other type of nucleotide and/or combination of types of nucleotides. The sample may include material or DNA isolated from two or more types of cells within a particular organism. Where the sample contains RNA, it may contain multiple different isoforms of a particular RNA sequence (e.g., relating to respective different isoforms of a folded RNA, protein generated from the RNA by a ribosome or other structure(s), or some other RNA-related substance).

The polynucleotide sequencer(s) may include any scientific instrument that performs polynucleotide sequencing (e.g., DNA sequencing, RNA sequencing) autonomously or semi-autonomously. Such a polynucleotide sequencer may receive a sample as an input, carry out steps to break down and analyze the sample, and generate read data representing sequences of read fragments of the polynucleotide(s) in the sample. A polynucleotide sequencer may subject DNA (or some other polynucleotide) from the sample to fragmentation and/or ligation to produce a set of polynucleotide fragments. The fragments may then be amplified (e.g., using polymerase chain reaction (PCR)) to produce copies of each polynucleotide fragment. Then, the polynucleotide sequencer may sequence the amplified polynucleotide fragments using, for example, imaging techniques that illuminate the fragments and measure the light reflecting off them to determine the nucleotide sequence of the fragments. Those nucleotide sequence reads may then be output as read data (e.g., a text file with the nucleotide sequence and other metadata) and stored onto a storage medium.

Read data alignment can include any combination of hardware and software that receives raw polynucleotide fragment read data and generates the aligned read data. In some embodiments, the read data is aligned to a reference genome (although, one or more nucleotides or segments of nucleotides within a read fragment may differ from the reference genome), to sequences corresponding to known structural variants from the reference genome that have been observed and/or that have been inferred from the read data. In some instances, the polynucleotide sequencer may also align the read fragments and output aligned read data.

Aligned read data may be any signal or data indicative of the read data and the manner in which each fragment in the read data is aligned (e.g., with a reference genome, with a sequence of a structural variant from the reference genome). An example data format of the aligned read data is the SAM format. A SAM file is a tab-delimited text file that includes sequence alignment data and associated metadata. Other data formats may also be used (e.g., pileup format).

B. Reference Genome

As described herein, a “reference genome” may refer to polynucleotide sequencing data and/or an associated predetermined nucleotide sequence for a particular sample. This could include DNA sequences (e.g., for the genomes of humans, plants, animals, bacteria, DNA viruses, etc.), RNA sequences (e.g., for the genomes of RNA viruses), or some other polynucleotide sequence of an organism of interest. A reference genome may also include information about the sample, such as its biopsy source, gender, species, phenotypic data, demographic data, and other characterizations. A reference genome may also be referred to as a “gold standard” or “platinum” genome, indicating a high confidence of the accuracy of the determined nucleotide sequence. An example reference genome is the NA12878 sample data and genome. In examples wherein the sample contains a synthetic DNA or other synthetic polynucleotide (e.g., samples wherein containing synthetic DNA used to store information in the sequence or other characteristics of the synthetic DNA), the reference genome could be a record of a baseline, unmodified, or otherwise reference state of the synthetic DNA in the sample.

C. Variant Types and Detection

As described herein, a genome may contain multiple chromosomes, each of which may include genes. Each gene may exist at a position on a chromosome referred to as the “gene locus.” Differences between genes (i.e., one or more variants at a particular gene locus) in different samples may be referred to as an allele. Collectively, a particular set of alleles in a sample may form the “genotype” of that sample.

Two genes, or, more generally, any nucleotide sequences that differ from each other (in terms of length, nucleotide bases, etc.) may include one or more variants. In some instances, a single sample may contain two different alleles at a particular gene locus; such variants may be referred to as “heterozygous” variants. Heterozygous variants may exist when a sample inherits one allele from one parent and a different allele from another parent; since diploid organisms (e.g., humans) inherit a copy of the same chromosome from each parent, variations likely exist between the two chromosomes. In other instances, a single sample may contain a gene that varies from a reference genome; such variants may be referred to as “homozygous” variants.

Many different types of variants may be present between two different alleles. Single nucleotide polymorphism (SNP) variants exist when two genes have different nucleotide bases at a particular location on the gene. Insertions or deletions (INDELs) exist between two genes when one gene contains a nucleotide sequence, while another gene contains a portion of that nucleotide sequence (with one or more nucleotide bases removed) and/or contains additional nucleotide bases (insertions). Structural differences can exist between two genes as well, such as duplications, inversions, and copy-number variations (CNVs).

Depending on the sensitivity and implementation of a variant caller, read data from a whole genome may include millions of potential variants. Some of these potential variants may be true variants (such as those described above), while others may be false positive detections. Such variants may correspond to candidate variant events in a database of known variants (e.g., known deletions, insertions, indels, transpositions, SNVs, tandem repeats, etc.).

### III. DETERMINATION OF A SAMPLE GENOME FROM A REFERENCE GENOME AND A SET OF READ FRAGMENTS

It is desirable in a variety of applications to unambiguously determine a sequence for DNA, RNA, or some other target polynucleotide in a sample. However, limitations in the cost and capability of current sequencing technologies, as well as the properties of target polynucleotides of interest and of the samples that contain the target polynucleotides, often result in fragmented or ambiguous reads from which it is difficult to determine the ‘true’ sequence of the target polynucleotide. This ambiguity in reconstruction can be related to the properties of the genome itself, as natural genomes often extensive repetition, inversion, translocation of sequences, or other features that can make it difficult to unambiguously determine the sequence of the genome from a set of experimentally-derived read fragments.

The systems and methods provided herein improve the process of reconstructing the sequence of a sample genome by repeatedly applying candidate variant events (e.g., deletion events) to a reference genome. Following application of a putative variant event to the in-process sample genome, available read fragments are applied to the in-process sample genome and a cost function evaluated. The cost function can be dependent on the number of the read fragments that are (or are not) mapped to the sample genome, a degree of correspondence between the distribution of the coverage of the sample genome by the read fragments and the expected distribution (e.g., Poisson, Gaussian, or some other specified reference distribution that is related to the expected coverage distribution given the fragmentation, priming, amplification, sequencing, and/or other processes used to generate the read fragments), demographic information about the applied variant event(s), or other factors. The applied variant event can then be retained or rejected, and the process repeated with another candidate variant event until a termination criterion is reached. Such an iterative method allows a sample genome to be accurately and efficiently reconstructed from read fragments by leveraging information about the reference genome and the expected or observed ways in which the sample genome is likely to differ from the reference genome, e.g., via a set of structural variants and/or other variant events.

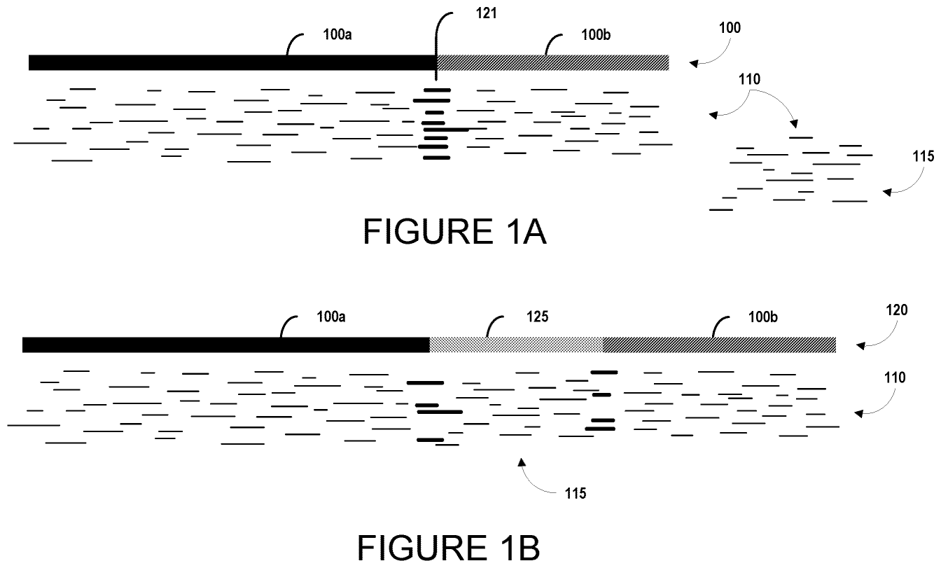

FIG. 1A illustrates, by way of a simplified schematic diagram, the alignment of a plurality of read fragments 110 to a reference genome 100. The reference genome 110 includes a first sequence 100a and a second sequence 100b that neighbor each other at a reference nucleotide location 121. As shown, a portion of the read fragments 115 have not been mapped to the reference genome. This could be due to there being no place on the reference genome 110 to which the unmapped fragments could be mapped and/or due to a quality of mapping of the unmapped fragments being less than a threshold level. Additionally, a subset of the mapped fragments (illustrated by thicker-weight lines) have been mapped such that they span the reference nucleotide location 121 and poorly map to one or the other of the first sequence 100a or the second sequence 100b.

The plurality of read fragments 110 represent respective portions of a sample genome of interest and may have been generated via a sequencing experiment (e.g., via a next-generation sequencing system or method) or via some other means applied to a biological sample taken from an individual (e.g., a human, animal, plant, or other organism). FIG. 1B illustrates, by way of a simplified schematic diagram, the alignment of the plurality of read fragments 110 to a sample genome 120 that represents the actual genome of the individual from which the read fragments 110 were generated. As shown, the sample genome 120 differs from the reference genome 100 in that an insertion sequence 125 has been inserted into the reference genome 100 at the reference nucleotide location 121. Thus, all of the plurality of read fragments 110 are able to be mapped to the sample genome 120, with the portion of the read fragments 115 that were not mapped to the reference genome 100 having been mapped to respective portions of the insertion sequence 125. Additionally, the read fragments that spanned the reference nucleotide location 121 (indicated by darker-weight lines) are better able to map to the sample genome because their portions that poorly mapped to one or the other of the first sequence 100a or the second sequence 100b now map to an appropriate portion of the insertion sequence 125.

In practice, a sample genome is likely to differ from a reference genome by more than a single insertion, as illustrated in FIGS. 1A and 1B. Instead, a sample genome is likely to differ from a reference genome by a number of insertions/deletions (e.g., novel sequence insertions), transpositions, inversions, copy number variations, tandem duplications/repeats, non-tandem or dispersed duplications/repeats, indels, inversions, translocations or other structural variants and/or smaller types of variants (e.g., single-nucleotide polymorphisms). Indeed, the variants may ‘overlap’ (e.g., a second insertion may be made at a location within a first insertion).

The variants represented in a sample genome may be truly novel, either in that they have not ever occurred before, or in that they have never been experimentally or clinically observed before. However, most of the variation within a given sample genome is likely to have been observed before, in the observed genomes of other individuals. Thus, the methods described herein can be used to efficiently reconstruct a sample genome by applying structural variants, from a database of candidate structural variants, to a reference genome. The database of candidate structural variants can include structural variants and/or single-nucleotide polymorphisms or other small variants that have been observed before. For example, the database of candidate structural variants could include structural variants that are included in the databases created by the Genome in a Box (GIAB) and/or Human Genome Structural Variation (HGSV) consortiums. Additionally or alternatively, the database of candidate structural variants can include variant events determined from a plurality of observed read fragments using other fragment alignment and variant calling methods, e.g., using the DeepVariant analysis pipeline.

The selection of which variant events to include in order to generate an accurate sample genome from a reference genome is a complex problem. This is because the effect of multiple different variant events on the sample genome (and thus on the potential patterns of alignment of read fragments thereto) can be highly dependent on each other. For example, an insertion event could be made into a region that includes a tandem repeat event. Both the relative locations of the insertion and tandem repeat as well as the order in which the events were applied to the reference genome to arrive at the sample genome will affect the sequence of the resulting sample genome. This event selection problem is further complicated by the need to map the read fragments to the sample genome in order to assess the ‘quality’ of the sample genome; the mapping of any particular read fragment can be ambiguous, especially where the read fragment may be mapped to a region of the sample genome that exhibits repeated sequences.

Nonlinear programming or other techniques can be used to perform variant event selection and/or refinement (e.g., determining the location, size, or other properties of variants that exhibit variation with respect to such properties). Additionally or alternatively, an iterative method may be used to repeatedly apply putative variant events to a reference genome, retaining those that result in an improved mapping of the read fragments while rejecting those that result in worse mapping, or that do not improve the mapping by at least a threshold amount.

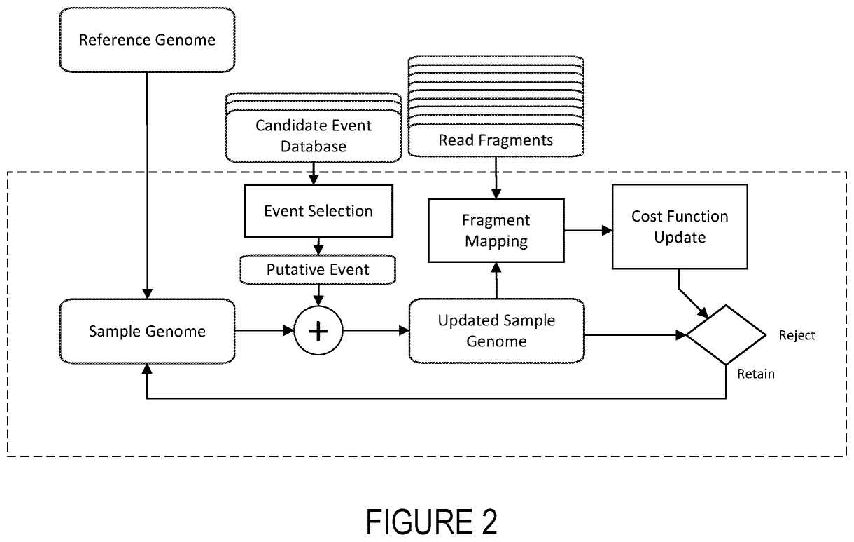

FIG. 2 illustrates a flowchart of elements of such a process for generating a sample genome from a reference genome and a plurality of read fragments by applying, to the reference genome, a set of variant events (e.g., insertions, deletions, inversions, translocations, copy number variants, etc.) selected from a database of candidate variant events. The portions of the process within the dashed line are performed repeatedly. A putative event is selected (via an “Event Selection” process) from a database of candidate events. The putative event is then applied to a sample genome (or to a reference genome, in the first repetition of the process) to generate an updated sample genome. The plurality of read fragments are then mapped to the updated sample genome (via a “Fragment Mapping” process) and this mapping is used to generate an update to a cost function that is indicative of the quality of the mapping of the plurality of read fragments to the updated sample genome. Based on the cost function update (which can include generating a cost function and/or merely generating a modification or other update thereto), the updated sample genome is either retained (in which case, additional variant events may be applied to the updated sample genome in additional repetitions of the process) or rejected. In either case, the applied putative event may be removed from the database of candidate events (either to remove it from future applications since it has already been applied, or to remove it from the possibility of re-evaluation for having been found wanting with respect to the cost function).

The reference genome may be obtained from a database or via some other means. For example, the reference genome may be generated based on extensive high-quality sequencing of one or more individuals of the same species. Putative events could then be repeatedly applied to the reference genome to iteratively generate, based on a set of read fragments from an individual, a sample genome for the individual. In such an example, the initial sample genome could simply be a copy of the reference genome, prior to application of any putative events thereto. Alternatively, a set of high-likelihood events could be applied to the reference genome in order to generate an initial sample genome, to which additional putative events could be applied. Such high-likelihood events could be selected from a database of candidate events based on, e.g., demographic information about an individual, phenotypic information about an individual (e.g., an observation that the individual exhibits a trait that is associated with a particular structural variant), prior genotyping information about an individual (e.g., simple gel-based fragment length information), the set of read fragments obtained from an individual (e.g., a set of events output by a machine-learning-trained model that receives the set of read fragments and/or a pattern of mapping thereof to the reference genome/candidate events), or based on some other factor(s).

Candidate events in the database of candidate events could be obtained in a variety of ways and/or from a variety of sources. For example, some or all of the candidate events could be structural variants or other patterns of variation contained within databases of genetic variation within a population of humans (or other organisms), e.g., databases created by the Genome in a Box (GIAB) and/or Human Genome Structural Variation (HGSV) consortiums. Additionally or alternatively, the me or all of the candidate events could be structural variants or other patterns of variation predicted, based on the set of read fragments and/or some other information source, by fragment alignment and/or variant calling methods, e.g., using the DeepVariant analysis pipeline.

Selection of an event to apply to the current sample genome estimate can include a variety of processes. In some examples, the selection could be wholly or partially random. For example, the probability of selection of a particular putative event from the database could be related to a probability that the particular event is present in a particular individual. Such a probability could be completely independent of additional information about the particular individual (e.g., a pure prior probability of the presence of the event in any individual of a population) or could be conditioned on information about the individual. For example, the probability could be based on demographic information about the individual, phenotypic information about the individual, etc.

Additionally or alternatively, the selection of an event to apply to the current sample genome estimate can include adding a set of events in a pre-determined order. Such a pre-determined order could be determined by assessing a degree of change in a cost function that would occur if each event was individually applied to the reference genome. The events could then be applied, via the process illustrated in FIG. 2, in order of decreasing the degree of change or according to some other ordering (e.g., randomly, with the probability of selection biased toward events having higher values of the degree of change in the cost function). Such degrees of change to the cost function could be calculated only once (e.g., prior to applying any events to the reference genome, or after applying a set of high-likelihood events to the reference genome but prior to applying additional events according to the process of FIG. 2). Alternatively, such degrees of change to the cost function could be evaluated multiple times (e.g., once after application and retention of each retained putative event) in order to update the order of application of additional putative events to account for the dependency of the cost function change on the other, already-applied events.

Applying a putative event to the sample genome can include modifying a computer record of the sample genome to generate the updated sample genome ‘in place’ in a computer memory. In such an implementation, later rejecting the applied putative event would require modifying the computer record again, to remove the effects of applying the putative event. Alternatively, applying a putative event to the sample genome can include modifying a copy of the sample genome to generate the updated sample genome in a location of a computer memory that is separate from the location where the un-altered sample genome is stored. In such an implementation, later retaining the applied putative event would require using the updated sample genome as the ‘input’ sample genome for subsequent repetitions of the process described in FIG. 2 (e.g., by setting a flag or pointer to indicate that the record containing the updated sample genome, and not the record containing the pre-event-application sample genome, is now the ‘current’ sample genome).

Selecting and applying a putative event can include determining an alignment, size, or other information about the putative event. This can include assessing a cost function (e.g., a degree of change in the cost function) for a variety of different alignments and/or sizes of the putative event and selecting a size and/or alignment that results in a maximal decrease in the cost function. Additionally or alternatively, some repetitions of the process of FIG. 2 could replace the selection and application of an additional putative event with a process of refinement of the size and/or alignment of one or more events that have already been applied to the sample genome.

Selecting and applying a putative event can include identifying a set of events from the database of candidate events, and an ordering therefor, and applying the identified set of events as a single “composite” putative event to a sample genome and/or to a reference genome. The identity and ordering of events within such a composite event could be determined using linear programming, nonlinear programming, integer linear programming, Markov chain Monte Carlo methods, or some other methods operating on the same cost function used to evaluate the effect of the composite effect on the sample genome or operating on some other cost function (e.g., a cost function that has been simplified, transformed, or otherwise modified to facilitate the applied selection method). The use of such methods could allow for the determination of sample genomes that are globally optimal (rather than merely locally optimal) by, e.g., running such methods to convergence. The use of such methods could also include, subsequent to inserting such a composite event into reference or sample genome, removing the composite event in favor of some other putative event (e.g., an alternative composite event generated by similar or different processes).

Mapping read fragments to locations on the updated sample genome can include a variety of processes. This can include performing dynamic programming methods (e.g., Needleman-Wunsch, Smith-Waterman), heuristic methods (e.g., FASTA, BLAST), or some other methods to map each read fragment in the plurality of read fragments to a location in the updated sample genome or to determine that a set of the read fragments do not map to the updated sample genome. Mapping the read fragments can be performed de novo, or may be partially pre-computed. For example, a set of potential mappings could be determined between each read fragment and one or more locations on the reference genome and/or one or more events in the candidate event database. Thereafter, mapping a particular read fragment to an updated sample genome could include selecting a mapping from the pre-computed set of one or more mappings for the particular read fragment.

Mapping read fragments to an updated sample genome could include determining groups of read fragments and mapping such groups, in whole or by portions, to events present in the updated sample genome and/or to portions of the reference genome represented in the updated sample genome. Such groups of read fragments could be identical or similar fragments that are difficult to uniquely map to respective locations within the updated sample genome due to, e.g., having been sequenced from repeat-containing regions or otherwise stereotyped regions of a subject's genome. Grouping the read fragments in this manner, and mapping them by proportion to candidate events rather than by individual alignments, can result in reduced computational cost to implement the mapping process. This is due, in part, to the fact that only a few proportion values assigning the group of read fragments in bulk to respective different candidate events need be determined, rather than a discrete alignment value for each of the read fragments. Further, grouping the read fragments in such a manner can also have other benefits, e.g., by allowing composite candidate events to be determined via blocked Gibbs sampling or some other integer linear programming (ILP), linear programming (LP), and Markov Chain Monte Carlo (MCMC), or other process(es).

The mapping of each fragment may include determining a flag for the fragments, e.g., a flag that indicates that the fragment is accurately mapped (e.g., more than a specified proportion of the fragment bases match bases in the updated sample genome), that the fragment is inaccurately mapped (e.g., less than a specified proportion of the fragment bases match bases in the updated sample genome, or a contiguous half of the fragment bases do not match bases in the updated sample genome), or that the fragment is unmapped to any location of the updated sample genome. Such flags may be used to determine a cost function and/or to determine an update to the cost function used to determine whether to retain or reject the applied putative event.

Updating a cost function based on the mapping of read fragments to the updated sample genome can include determining a variety of properties related to the read fragments, their alignment (or lack of alignment) to the updated sample genome, the set of events already applied to the sample genome and/or the putative event most recently applied to the sample genome, the demographics or phenotype of an individual from whom the read fragments were generated, and/or related to some other relevant properties.

The cost function could include one or more terms related to the number or proportion of the read fragments that are mapped to the updated sample genome, the number or proportion of the read fragments that are not mapped to the updated sample genome, and/or the quality or pattern of mapping of the mapped read fragments to the updated sample genome. For example, a Smith-Waterman score could be determined for each read fragment, relative to the updated sample genome, and read fragments having scores below a specified threshold level could be considered un-mapped (and thus not included in determining the coverage distribution for the updated sample genome). In some examples, multiple different sub-sequences within a single read fragment could be mapped to multiple different locations within the updated sample genome and/or unmapped (e.g., due to spanning a breakpoint for a known structural variant event). Such a read fragment may be referred to as a “splitting read” and could be counted as mapped, unmapped, or partially mapped; the appropriate portion(s) of such a splitting read could contribute to the coverage distribution of the portion(s) of the updated sample genome to which it is mapped. In some examples, there could be relationships between pairs (or other sets) of the read fragments, and the cost function could include terms related to information about such relationships. For example, the read fragments could be generated via a paired-end NGS technique, in which case information about the relative orientation and spacing of the pairs of read fragments would be known. Accordingly, the cost function could include one or more terms related to the concordance (or lack thereof) between the known information about a pair of mapped read fragments and their relationship as mapped to an updated sample genome. Pairs of read fragments whose mapping is discordant with the known information about their relative orientation and/or spacing may be referred to as “discordant read-pairs,” and a cost function could include a term relating to the number or proportion of such discordant read-pairs in a mapping of the read fragments to the updated sample genome.

The cost function could include one or more terms related to the distribution of coverage of the updated sample genome by the read fragments. In many cases, it is expected that the distribution of coverage of a reconstructed genome by observed read fragments will approximate a theoretical distribution (e.g., Poisson, Gaussian, etc.) having properties related to the method used to generate the read fragments (e.g., the fragmentation, priming, amplification, sequencing, and/or other processes used to generate the read fragments). For example, in many cases it is expected that the coverage distribution will comport with a Poisson distribution having a mean related to the sequencing depth of the process used to generate the read fragments. The degree of correspondence could be represented, in the cost function, by a term related to the Chi-squared test statistic comparing the observed coverage distribution to the theoretical coverage distribution. A coverage distribution term in the cost function could be determined and/or updated based on the global coverage distribution for an updated sample genome and/or based on the local distribution (e.g., the coverage distribution in the neighborhood of an inserted putative event). The objective term or “fitness” for the correspondence of the coverage distribution to the Poisson distribution Pois(x; λ) could be formulated as:

$$F ( E , λ ) = {\sum }_{i = 1}^{N} log Pois ( {c}_{i}^{E} ; λ ) - N \cdot μ ( λ )$$

where E is the set of inserted events, N is the length of the constructed genome G^{E}, cᵢ^{E} is the read coverage at position i along the constructed genome, λ is the sequencing depth, and μ(λ) is the expected log score for each position depending on the sequencing depth λ. This optimization objective (cost function) may be viewed as a ratio of a score for the coverage distribution from the constructed genome for the set of inserted events to an expected coverage score for a coverage distribution of the same length N. Such a distribution-related cost function could be adjusted to take into account the observation that un-mapped read fragments generally will not map to un-applied events in a candidate event set if those un-applied events are, in fact, not present in the source individual's genome. Such a corrected cost function term or “fitness” could be formulated as:

F=F(E,λ)+F(Ē,1)
with F(Ē, 1) being the above formulation computed, instead, across the sequences of the set of non-applied events Ē using only un-mapped read fragments that can be mapped to events in the set of non-applied events Ē.

The cost function could include one or more terms related to the demographics and/or phenotype of the individual from which the read fragments were generated. For example, the cost function could include one or more terms related to the likelihood of observing the putative event and/or the combination of the applied events in the updated sample genome given the demographics of the individual and/or observed phenotypic trait(s) of the individual.

The cost function could include one or more terms related to statistical structure in the co-occurrence of certain known variant events. For example, a certain putative event could be more likely to occur in combination with one or more other candidate events. In such an example, the cost function could include a term relating to the increased likelihood of the putative event being present in the updated sample genome if one or more of the other candidate events have already been applied to the updated sample genome. Such a cost function term could be formulated as a conditional probability of observation of the putative event given the presence of the set of candidate events already applied to the updated sample genome prior to applying the putative event.

The cost function could include one or more terms intended to penalize over-fitting or to otherwise encourage parsimonious event sets in describing the variation of a sample genome relative to a reference genome. For example, the cost function could include a term related to the number of events applied to the reference genome to arrive at the updated sample genome. The cost function could include a term that compensates for the ability of longer structural variants (e.g., insertions) that are not present in an underlying sample genome to, nonetheless, artefactually increase the number of “mapped” reads due merely to the additional length of the sequence of the event.

Updating the cost function could include re-computing the cost function based on the mapping of the entire set of read fragments to the updated sample genome. In practice, however, applying a putative event to a sample genome will only result in updating the mapping of a small subset of the read fragments. Such fragments can include those read fragments mapped at or near the location of the applied event within the updated sample genome and/or those read fragments that were previously mapped to other locations of the sample genome prior to applying the applied event that are now mapped at or near the location of the applied event. Thus, it can be more efficient, with respect to computational cost and/or memory use, to update the cost function by computing a change in the cost function due to changes in the neighborhood of the applied event and/or due to the remapping of read fragments from other regions of the sample genome to the neighborhood of the applied event. For example, updating the cost function could include computing a change in the cost function, the change in the cost function computed based on the changes in the cost-function-related factors in the neighborhood of the applied event and/or in neighborhoods of the sample genome to which read fragments were formerly mapped but that were re-mapped to the neighborhood of the applied event.

The cost function update could also be used to determine when to terminate the repeated process of addition of events to the sample genome. This could include terminating the process due to the cost function update representing a total cost function value that is greater than a threshold value, or the cost function update representing a sub-threshold improvement of the overall cost function (or such a sub-threshold improvement for more than a specified number of repetitions). Additionally or alternatively, the repeated process could be terminated due to a specified time or compute cost having been spent, a specified number of events having been added to the reference genome, a specified proportion of the read fragments having been mapped to the updated sample genome, and/or some other termination criterion.

Note that a variety of improvements are possible to the process depicted in FIG. 2 and/or to sub-processes thereof. For example, instead of a simple repetitive process of addition of events to the reference genome, nonlinear programming or other techniques could be used to determine the selection, ordering, or other properties of the set of events applied to a reference genome to generate a sample genome for an individual. Such techniques include integer linear programming (ILP), linear programming (LP), and Markov Chain Monte Carlo (MCMC).

In some examples, determining alignments between the read fragments and the sample genome and/or updated sample genome could include generating a population reference graph by merging the reference genome and the inserted events and then using the population reference graph to align the read fragments.

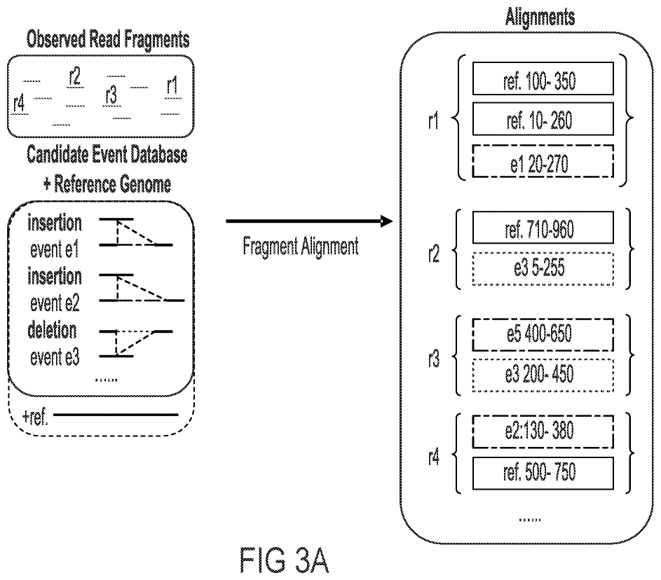

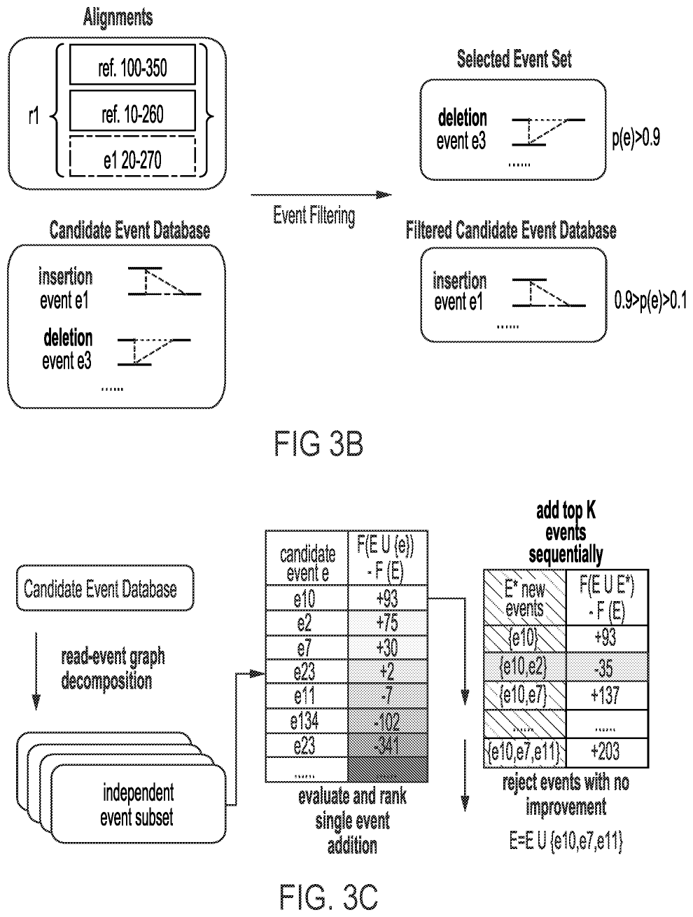

In some examples, a set of possible alignments to the reference genome and/or one or more events in a candidate event database could be pre-computed for each observed read fragment. By pre-computing the alignments, the iterative process of re-mapping the read fragments to the sample genome following application of a putative event can be reduced to selection of the appropriate alignment for each read fragment from a small list of possible alignments. Aspects of such a pre-alignment process is illustrated by way of example in FIG. 3A. So, three possible alignments are determined for a first read fragment, r1, to two different locations on the reference genome (to reference nucleotide positions 100-350 and 10-260) and to a location on a first event e1 (to nucleotide positions 20-270 on event e1). In another example, two possible alignments are determined for a third read fragment, r3, to a location on a fifth event e5 (to nucleotide positions 400-650 on event e5) and to a location on a third event e3 (to nucleotide positions 200-450 on event e3). Note that a particular read may map only partially to one or more portions of a particular event or reference genome (e.g., due to the read fragment being a “splitting read” that maps across a breakpoint between a structural variant and a reference genome or between two different structural variants). Selection between different possible alignments could be determined based on a Smith-Waterson score or some other measure of the quality of the alignment of the read fragment to a corresponding portion of an updated sample genome.

Such pre-alignment of read fragments can provide additional benefits. For example, the overall task of generating the sample genome can be separated into independent sub-problems based on the set of pre-alignments. This can be done by analyzing the set of pre-alignments to determine independent sets of read fragments and candidate events, e.g., by treating the set of pre-alignments as a read-event graph representing the structure of dependencies between the events and the read fragments. In such an independent set of read fragments and candidate events, the read fragments only have possible mappings to locations in the candidate events and to locations in the reference genome that are not affected by any other candidate events. Additionally, the candidate events in such an independent set of read fragments and candidate events also only map to read fragments in the independent set. Separating the problem into such sub-problems simplifies the calculation of cost functions, as information relevant to the selection, retention, or rejection of any candidate event in the sub-problem is limited to the neighborhoods of the sample genome near the location of the candidate events and/or locations on the reference genome corresponding to the pre-alignments of the read fragments in the sub-problem. Additionally, each sub-problem can be allocated to a respective different processor, server, virtual machine, or other separate computing substrate, allowing for increased efficiency and reduced cost. Yet further, separating the overall event selection problem into a number of smaller, independent sub-problems can make the application of nonlinear programming techniques more tractable. Indeed, separating the overall event selection problem into a number of smaller, independent sub-problems could, in some instances, allow for a complete sampling of the space of possible candidate event selections.

In some examples, a set of candidate events could be pre-filtered in order to simplify, speed, or otherwise improve the process of selecting and applying the events to a reference genome in order to arrive at a sample genome for an individual. This could include filtering the set of candidate events to remove those events that are especially unlikely to be present in a sample genome, or to apply a set of candidate events that are especially likely to be present in a sample genome to a reference genome in a single step prior to repeatedly applying additional candidate events.

FIG. 3B illustrates such a filtering process, where a set of pre-computed alignments between read fragments are used to filter events in a candidate event database into a ‘selected event database’ (which contains events evaluated to have a probability of inclusion in a sample genome of greater than a threshold level of 0.9) and a ‘filtered candidate event database’ (which contains events evaluated to have a probability of inclusion in a sample genome of less than a threshold level of 0.9 but greater than a threshold level of 0.1). Events from the ‘selected event set,’ being highly likely to be present in the sample genome of the individual from whom the read fragments were generated, are applied to the reference genome to generate an initial estimate of the individual's sample genome. The repeated process of FIG. 2, or some other process (e.g., nonlinear programming) is then used to add additional events from the ‘filtered candidate event database’ to this initial estimated genome to arrive at a final estimated sample genome for the individual.

A variety of filtering methods could be used to extract from a set of candidate events one or more sets of likely, unlikely, and/or highly likely events for further processing in determination of a sample genome. For example, a machine learning classifier could be trained to receive demographic data, pre-computed read-event graphs, local read features of inserted events (e.g., sequence coverage, paired-read insert distributions, split-reads), or other information and to output a prediction of the likelihood that a particular event is present in a sample genome. Such a machine learning classifier could be trained based on real, observed sets of read fragments and sample genomes and/or based on simulated genomes and read fragments. The output of such a machine learning classifier filter could be a binary output (representing, e.g., that a particular candidate event is present or absent) or could be a confidence level between zero and one. In examples wherein the output is a confidence level, the confidence level could be used to sample potential candidate events as part of the iterative reconstruction process described herein, e.g., a candidate event with a higher predicted confidence level could be more likely to be sampled and inserted into a putative genome than an alternative candidate event with a lower predicted confidence level. Such a machine learning classifier filter could also provide output(s) indicative of the zygosity of candidate events, e.g., that a particular candidate event is likely to have occurred in a maternal copy of a gene/chromosome, in a paternal copy of a gene/chromosome, or both copies of a gene/chromosome.

Additionally or alternatively, a variety of heuristic filtering methods could be applied. This could include identifying the existence of “signature reads” within the set of read fragments obtained from an individual. Such “signature reads” are reads that indicate, with high probability, that a particular variant event is or is not present in the sample genome of the individual. A “positive” signature read for a particular variant event is a read fragment whose existence makes it highly likely that the particular variant event is represented in the sample genome. This may be due to the sequence of the positive signature read being extremely unlikely to be observed unless the particular variant event is present (e.g., due to the positive signature read being especially long, the sequence of the positive signature read being especially unique, and/or the sequence of the positive signature read being especially unlikely to occur due to errors in the sequencing process as a result of the presence of some other event or reference sequence present in the individual's genome). A “negative” signature read for a particular variant event is a read fragment whose existence makes it highly unlikely that the particular variant event is represented in the sample genome. This may be due to the sequence of the negative signature read being extremely unlikely to be observed if the particular variant event is present (e.g., due to the negative signature read being especially long, the sequence of the negative signature read being especially unique, the sequence of the negative signature read being especially unique to the event's alternative reference sequence, the sequence of the negative signature read corresponding to an alternative event that does not co-occur with the particular variant event, and/or the sequence of the negative signature read being especially unlikely to occur due to errors in the sequencing process as a result of the presence of the particular variant event within the individual's genome).

FIG. 3C illustrates, by way of example, a number of improvements to the process illustrated in FIG. 2. A candidate event database (which may have been pre-filtered to remove especially low and/or high likelihood events) is decomposed into a number of independent event subsets based on a read-graph event decomposition based on potential mappings between observed read fragments and the events in the candidate event database. This decomposition into independent subsets of events allows each independent event subset to be evaluated independently, e.g., on different servers, different virtual machines, etc. Such decomposition can also relax the memory and compute requirements of the event selection and assessment process. Further, decomposition of the event selection process into smaller subsets of events can allow different selection processes to be applied (e.g., an exhaustive search of the event selection space within each independent event subset) and/or improve the outcome of applied selection methods (e.g., allow a nonlinear programming algorithm to select, with higher confidence, a globally optimal set of events from each independent event subset).

As shown in FIG. 3C, the effect on a cost function of adding each single candidate event is calculated. This is depicted in FIG. 3C as “F(E∪{e))−F(E)” to indicate that the effect on the cost function is calculated as a differential cost function i.e. the differential fitness for events E and candidate event e. These effects, within each independent event subset, are then ranked (“evaluate and rank single event addition”). As shown, the most beneficial events (e.g., “e10”) result in the largest positive differential cost, while the least beneficial events (e.g., “e23”) result in the largest negative differential cost. However, this is a result of the specific cost function used, and the most beneficial events could, instead, result in the largest negative differential cost (in such an example, the cost function could instead be referred to as an objective function).

The events are then applied according to the ranked differential cost function values using the repeated process of FIG. 2. The cost function update determined to assess each applied event is the differential cost function between the cost function with the additional applied event present and the cost function with the additional applied event present. This is depicted in FIG. 3C as “F(E∪E*)−F(E),” with E being the set of previously-applied events (the set of no events, in the case of the highest-ranked event in the independent subset) and E* being the most recently applied event, which is being assessed for retention or rejection. This differential cost function is then used to determine whether to reject or retain the most recently applied event. This can include comparing the differential cost function to a threshold differential cost function value.

As shown in FIG. 3C, this can include rejecting those events whose addition does not improve the cost function, i.e., whose addition does not result in a positive differential cost function. As illustrated in FIG. 3C, this means retaining event ‘e10’ since the differential cost function from its addition is +95 (necessarily identical to the single-event addition calculated for e10 in order to rank the events for order of assessment). However, the subsequent addition of event ‘e2’ (resulting in an applied event set of {e10,e2}) results in a negative differential cost function (−35), so event ‘e2’ is rejected. Addition of event ‘e7’ results in a positive differential cost function (+137), so event ‘e7’ is retained. Note that the differential cost functions for adding events ‘e2‘ or’e7‘ when event’e10’ has already been applied differ from the single-event differential cost functions for those events; this is due to the dependencies between the events in their effect on the cost function. The process is repeated, eventually resulting in the addition of events ‘e10,’ ‘e7,’ and ‘e11’ from the illustrated independent event subset to the reference genome. These events are added to the events selected from the other independent event subsets to result in the complete set of events added to the reference genome to result in a sample genome for an individual.

### IV. EXAMPLE METHODS

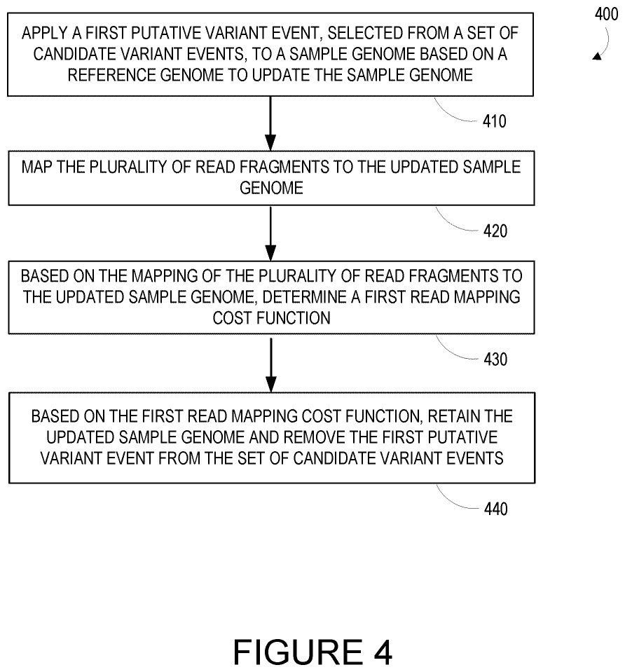

FIG. 4 depicts an example method 400 for determining a sample genome from a plurality of read fragments and a reference genome. The method 400 includes applying a first putative variant event, selected from a set of candidate variant events, to a sample genome based on the reference genome to update the sample genome (410). The method 400 additionally includes mapping the plurality of read fragments to the updated sample genome (420). The method 400 also includes, based on the mapping of the plurality of read fragments to the updated sample genome, determining a first read mapping cost function (430). The method yet further includes, based on the first read mapping cost function, retaining the updated sample genome and removing the first putative variant event from the set of candidate variant events (440). The method 400 could include additional steps or features.

In more detail, one example method may first perform a pre-alignment process as described above with reference to FIG. 3A. This may, for example, find (all) possible alignments between the observed read fragments and the reference genome, as well as the sequences of candidate structural variant events. In some implementations the candidate event sequences include context sequences before and after the events, and these may have the same length as the read fragments to facilitate aligning read fragments to events as if the events had already been introduced into the reference genome. A result of this process may be a map between read fragments and (all) their possible alignment positions in the reference genome and in the sequences of the candidate structural variant events. Such a map facilitates calculating coverage and other features. The possible alignments (which may be referred to as “the map”) may then be filtered as described above with reference to FIG. 3B, to identify likely candidates and prune unlikely candidates. Independently of this the possible alignments (the map) may be filtered using positive and/or negative signature reads as previously described, i.e. to include event(s), and/or to exclude counter-factual event(s), which have an essentially unique signature for their occurrence/nonoccurrence.

The selected events may then be incorporated into the reference genome (or into a previous version of the sample genome) to construct a candidate sample genome G^{E}. Possible alignments of the read fragments to G^{E} may then be sampled from the pre-alignments, resulting in a coverage map C^{E} defining the read coverage cᵢ^{E} at positions along G^{E} as previously described. A value for the fitness optimization objective (first read mapping cost function) may then be calculated e.g. by calculating a value of F(E, λ) or F as described above. An optimization process may then be applied to add or remove events to or from the sample genome to optimize the fitness optimization objective.

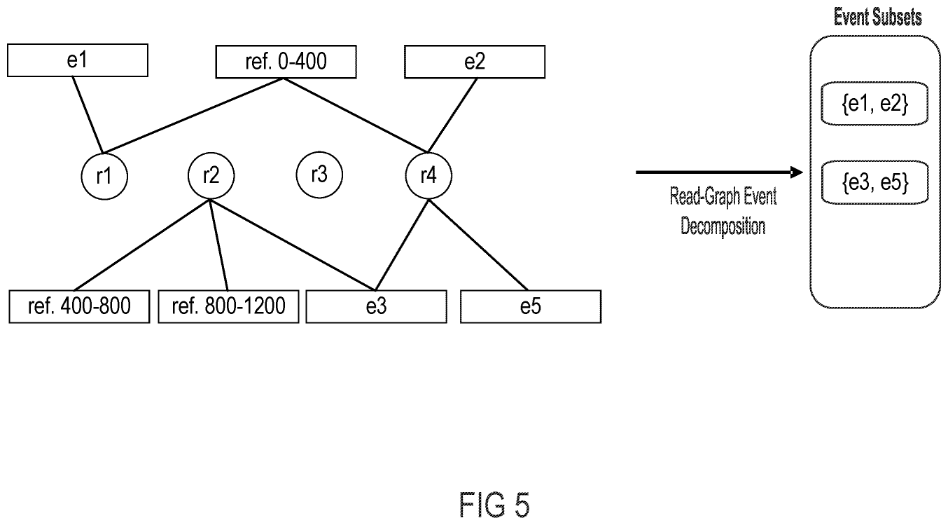

Read fragments may align to multiple different events and thus selecting events independently from one another could lead to suboptimal solutions. However, the number of possible event subsets is too large to cost-effectively explore. However the events (event database) can be decomposed into smaller independent subsets. For example sampling of alignments for an event that does not share multi-mapped reads with any other candidate events does not impact sampling of alignments of any other events (and vice-versa), and such events can thus be solved independently. This logic can be extended to “clusters” of events, by creating a graph with nodes comprising events, reference (base) intervals, and multi-mapped read fragments, and with edges between read fragments and the event(s) and/or reference intervals they align to. FIG. 5 shows an example event decomposition graph, which may be constructed starting from the result of the pre-alignment process. Events in different, unconnected clusters may be treated independently (and processed in parallel).

The optimization process of FIG. 3C may be performed within each cluster or subset of candidate events, e.g. in parallel. Thus candidate events may be ranked by their fitness relative to the current event selection, and the top K selected; e.g. K=25. Each may be tried in turn, only retaining the event if the fitness optimization objective is improved. The ranking may be repeated after adding a candidate, as this may affect the ranking. This may be repeated until there is no further improvement (greater than a threshold) and/or until a maximum number of iterations.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### V. CONCLUSION

It should be understood that arrangements described herein are for purposes of example only. As such, those skilled in the art will appreciate that other arrangements and other elements (e.g. machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

1. A computer-implemented method for determining a sample genome from a plurality of read fragments and a reference genome, the method comprising:
applying a first putative variant event, selected from a set of candidate variant events, to a sample genome based on the reference genome to update the sample genome;
mapping the plurality of read fragments to the updated sample genome;
based on the mapping of the plurality of read fragments to the updated sample genome, determining a first read mapping cost function; and
based on the first read mapping cost function, retaining the updated sample genome and removing the first putative variant event from the set of candidate variant events.

2. The computer-implemented method of claim 1, wherein the set of candidate variant events includes events from a database of experimentally observed structural variations.

3. The computer-implemented method of claim 1, wherein the set of candidate variant events includes at least one variant event determined from the plurality of read fragments.

4. The computer-implemented method of claim 1, further comprising, subsequent to retaining the updated sample genome and removing the first putative variant event from the set of candidate variant events:
applying a second putative variant event, selected from the set of candidate variant events, to the updated sample genome to further update the sample genome;
mapping the plurality of read fragments to the further updated sample genome;
based on the mapping of the plurality of read fragments to the further updated sample genome, determining a second read mapping cost function; and
based on the second read mapping cost function, rejecting the further updated sample genome.

5. The computer-implemented method of claim 4, wherein determining the second read mapping cost function comprises evaluating a cost function that includes a term that is related to at least one of: (i) a likelihood of the second putative variant event appearing in a demographic group that includes a subject from which the read fragments were obtained, or (ii) a likelihood of the likelihood of the second putative variant event appearing in a genome given that the first putative variant event appears in the genome.

6. The computer-implemented method of claim 1, further comprising, subsequent to retaining the updated sample genome and removing the first putative variant event from the set of candidate variant events:
updating at least one of an alignment or a size of the first putative variant event within the updated sample genome to further update the sample genome;
mapping the plurality of read fragments to the further updated sample genome;
based on the mapping of the plurality of read fragments to the further updated sample genome, determining a third read mapping cost function; and
based on the third read mapping cost function, retaining the further updated sample genome.

7. The computer-implemented method of claim 1, wherein determining the first read mapping cost function comprises determining, for at least one region of the updated sample genome, a degree of correspondence between a specified reference distribution and a distribution of coverage of the updated sample genome by the plurality of read fragments after mapping the plurality of read fragments to the updated sample genome.

8. The computer-implemented method of claim 1, wherein determining the first read mapping cost function comprises determining a number of read fragments of the plurality of read fragments that are not mapped to the updated sample genome after mapping the plurality of read fragments to the updated sample genome.

9. The computer-implemented method of claim 1, wherein determining the first read mapping cost function comprises determining a number of read fragments of the plurality of read fragments that are partially mapped to at least one location within the updated sample genome after mapping the plurality of read fragments to the updated sample genome.

10. The computer-implemented method of claim 1, wherein determining the first read mapping cost function comprises evaluating a cost function that includes at least one of: (i) a term that is related to the number of variant events applied to the reference genome to generate the updated sample genome or (ii) a term that is related to a number of read fragments that are mapped to an event from the set of candidate variant events that is not represented in the sample genome.

11. The computer-implemented method of claim 1, wherein determining the first read mapping cost function comprises determining, based on a region of the updated sample genome that is affected by application of the first variant event, a local effect on a global read mapping cost function.

12. The computer-implemented method of claim 1, further comprising:
prior to applying the first putative variant event to the sample genome to update the sample genome, determining a set of pre-alignments for the plurality of read fragments by determining one or more possible alignments for each read fragment to one or more locations on the reference genome or to one or more locations on one or more of the variant events in the set of candidate variant events,
wherein mapping the plurality of read fragments to the updated sample genome comprises selecting, for a particular read fragment in the plurality of read fragments, one of the possible alignments for the particular read fragment.

13. The computer-implemented method of claim 12, further comprising:
determining, based on the set of pre-alignments, that a first set of read fragments within the plurality of read fragments and a first set of variant events within the set of candidate variant events are connected via possible alignments to each other but not to other read fragments within the plurality of read fragments or to other variant events within the set of candidate variant events;
wherein the first putative variant event is selected from the first set of variant events, and wherein mapping the plurality of read fragments to the updated sample genome comprises mapping the first set of read fragments to the updated sample genome.

14. The computer-implemented method of claim 12, further comprising:
prior to applying the first putative variant event to the sample genome to update the sample genome, removing from the set of candidate variant events any variant events for which the set of pre-alignments includes no possible alignments with any read fragments.

15. The computer-implemented method of claim 1, further comprising:
applying a filter to determine a second set of variant events that are highly likely to be represented in the sample genome and a third set of variant events that are highly unlikely to be represented in the sample genome;
prior to applying the first putative variant event to the sample genome to update the sample genome, applying the variant events of the second set of variant events to the reference genome to generate the sample genome; and
prior to applying the first putative variant event to the sample genome to update the sample genome, removing the third set of variant events from the set of candidate variant events.

16. The computer-implemented method of claim 15, wherein applying the filter to determine the second and third sets of variant events comprises determining, for each of the variant events in the set of candidate variant events, at least one of: (i) the existence of a positive signature read within the plurality of signature reads, or (ii) the existence of a negative signature read within the plurality of signature reads, wherein determining the existence of a positive signature read within the plurality of signature reads for a particular variant event comprises determining that there exists, within the plurality of read fragments, a read fragment whose existence makes it highly likely that the particular variant event is represented in the sample genome, and wherein determining the existence of a negative signature read within the plurality of signature reads for a particular variant event comprises determining that there exists, within the plurality of read fragments, a read fragment whose existence makes it highly likely that the particular variant event is not represented in the sample genome.

17. A non-transitory computer readable medium having stored therein instructions executable by a computing device to cause the computing device to determine a sample genome according to a method comprising:
applying a first putative variant event, selected from a set of candidate variant events, to a sample genome based on the reference genome to update the sample genome;
mapping the plurality of read fragments to the updated sample genome;
based on the mapping of the plurality of read fragments to the updated sample genome, determining a first read mapping cost function; and
based on the first read mapping cost function, retaining the updated sample genome and removing the first putative variant event from the set of candidate variant events.

18. (canceled)

19. The non-transitory computer readable medium of claim 17, wherein the method further comprises, subsequent to retaining the updated sample genome and removing the first putative variant event from the set of candidate variant events:
applying a second putative variant event, selected from the set of candidate variant events, to the updated sample genome to further update the sample genome;
mapping the plurality of read fragments to the further updated sample genome;
based on the mapping of the plurality of read fragments to the further updated sample genome, determining a second read mapping cost function; and
based on the second read mapping cost function, rejecting the further updated sample genome.

20. The non-transitory computer readable medium of claim 17, wherein the method further comprises, subsequent to retaining the updated sample genome and removing the first putative variant event from the set of candidate variant events:
updating at least one of an alignment or a size of the first putative variant event within the updated sample genome to further update the sample genome;
mapping the plurality of read fragments to the further updated sample genome;
based on the mapping of the plurality of read fragments to the further updated sample genome, determining a third read mapping cost function; and
based on the third read mapping cost function, retaining the further updated sample genome.

21. The non-transitory computer readable medium of claim 17, wherein the method further comprises:
prior to applying the first putative variant event to the sample genome to update the sample genome, determining a set of pre-alignments for the plurality of read fragments by determining one or more possible alignments for each read fragment to one or more locations on the reference genome or to one or more locations on one or more of the variant events in the set of candidate variant events,
wherein mapping the plurality of read fragments to the updated sample genome comprises selecting, for a particular read fragment in the plurality of read fragments, one of the possible alignments for the particular read fragment.
